(19) United States
(12) Patent Application Publication
(10) Pub. No.: US 2023/0017647 A1
(43) Pub. Date: Jan. 19, 2023

(54) PUNCTURE NEEDLE, CATHETER ASSEMBLY, AND VASCULAR PUNCTURE SYSTEM

(71) Applicant: TERUMO KABUSHIKI KAISHA, Tokyo (JP)

(72) Inventors: Shota YAMAMOTO, Mitaka-shi (JP); Takayuki YOKOTA, Chuo-shi (JP)

(73) Assignee: TERUMO KABUSHIKI KAISHA, Tokyo (JP)

(21) Appl. No.: 17/947,342

(22) Filed: Sep. 19, 2022

Related U.S. Application Data
(63) Continuation of application No. PCT/JP2021/009832, filed on Mar. 11, 2021.

(30) Foreign Application Priority Data
Mar. 23, 2020 (JP) 2020-050645

(51) Int. Cl. A61M 5/158; A61M 25/06

(52) U.S. Cl. CPC A61M 5/158; A61M 25/06

(57) ABSTRACT

A medical puncture needle includes: a metal needle body formed in a tubular shape, the needle body including: a blade surface located at a distal end portion of the needle body, a planar reflection portion located at an inner surface of the needle body and configured to reflect light, and a transmission window located proximal of the blade surface and configured to transmit reflected light reflected by the planar reflection portion.

### CROSS-REFERENCE TO RELATED APPLICATIONS

This is a bypass continuation of PCT Application No. PCT/JP2021/009832, filed on Mar. 11, 2021, which claims priority to Japanese Application No. 2020-050645, filed on Mar. 23, 2020. The contents of these applications are hereby incorporated by reference in their entireties.

### BACKGROUND

The present disclosure relates to a puncture needle, a catheter assembly, and a vascular puncture system.

A puncture needle such as an indwelling needle includes, for example, a metal needle body formed in a tubular shape (see JP 2009-233007 A). In addition, in recent years, a technique for visualizing the travel of a blood vessel in a living body by an image obtained by receiving reflected light of near-infrared light with which the living body is irradiated has been developed.

### SUMMARY

Meanwhile, the metal needle body reflects near-infrared light, and the blood in the blood vessel absorbs near-infrared light. Therefore, the positional relationship between the needle body and the blood vessel in the puncture target site can be visualized by the image (reflected light image) obtained by receiving the reflected light of the light (for example, near-infrared light) with which the living body (puncture target site) punctured with the needle body is irradiated. However, the reflected light image indicates the planar positional relationship between the needle body and the blood vessel, and does not indicate the positional relationship between the needle body and the blood vessel in the depth direction. Therefore, the user cannot be aware of whether the needle body has secured the blood vessel based on the reflected light image.

Embodiments of the present invention have been developed in view of such problems, and an object of the certain embodiments is to provide a puncture needle, a catheter assembly, and a vascular puncture system capable of allowing for recognition that a blood vessel has been secured by a needle body based on a reflected light image.

According to a first aspect of the present invention a medical puncture needle includes a metal needle body formed in a tubular shape, in which the needle body includes a blade surface formed at a distal end portion of the needle body, a planar reflection portion provided at an inner surface of the needle body and configured to reflect light, and a transmission window capable of transmitting reflected light reflected by the planar reflection portion, and the transmission window is located on a proximal end side relative to the blade surface.

According to a second aspect of the present invention, a catheter assembly includes the above-described puncture needle and a catheter shaft having a lumen through which the needle body is inserted.

According to a third aspect of the present invention, a vascular puncture system includes the above-described puncture needle, an irradiation unit configured to irradiate a puncture target site punctured with the needle body with the light, and a light receiving unit configured to receive reflected light reflected by the puncture target site and the needle body.

According to certain embodiments the present invention, the light with which the puncture target site punctured with the needle body is irradiated is guided to the lumen of the needle body through the distal end opening, the proximal end opening, or the transmission window of the needle body. When the needle body is in the blood vessel unsecured state and the blood does not flow into the lumen of the needle body, the light guided to the lumen of the needle body is reflected by the planar reflection portion. The reflected light from the planar reflection portion is transmitted through the transmission window and is led to the outside of the needle body. Therefore, the user can visually recognize the transmission window in the needle body in the reflected light image. On the other hand, when the needle body is in the blood vessel secured state and the blood flowing into the lumen of the needle body covers the transmission window, the light guided to the lumen of the needle body is absorbed by the blood and thus is not led from the transmission window to the outside of the needle body. Therefore, the appearance of the transmission window of the needle body changes in the reflected light image. Therefore, the user can recognize the securing of the blood vessel of the needle body based on the reflected light image.

### BRIEF DESCRIPTION OF DRAWINGS

FIG. 1 is a schematic configuration diagram of a vascular puncture system according to an embodiment of the present invention;

FIG. 2 is an exploded perspective view of the catheter assembly of FIG. 1;

FIG. 3A is a plan view of a distal end portion of a needle body of FIG. 2, FIG. 3B is a cross-sectional view taken along line IIIB-IIIB of FIG. 3A, and FIG. 3C is a cross-sectional view taken along line IIIC-IIIC of FIG. 3B;

FIG. 4A is a first explanatory view of a method for forming a planar reflection portion in FIG. 3A, and FIG. 4B is a second explanatory view of the method for forming the planar reflection portion in FIG. 3A;

FIG. 5 is a first explanatory view of a puncture procedure for a blood vessel with the catheter assembly of FIG. 1;

FIG. 6 is a reflected light image in the state of FIG. 5;

FIG. 7 is a second explanatory view of a puncture procedure for a blood vessel with the catheter assembly of FIG. 1;

FIG. 8 is a reflected light image in the state of FIG. 7;

FIG. 9A is a transverse cross-sectional view of a needle body according to a first modification, and FIG. 9B is an explanatory view of a method of forming the planar reflection portion in FIG. 9A;

FIG. 10A is a transverse cross-sectional view of a needle body according to a second modification, and FIG. 10B is an explanatory view of a method of forming the planar reflection portion in FIG. 10A;

FIG. 11A is a transverse cross-sectional view of a needle body according to a third modification, FIG. 11B is a partially omitted longitudinal cross-sectional view of a needle body according to a fourth modification, and FIG. 11C is a partially omitted longitudinal cross-sectional view of a needle body according to a fifth modification;

FIG. 12 is a plan view of a needle body according to a sixth modification;

FIG. 13 is an explanatory view of a puncture procedure for a blood vessel with a catheter assembly including the needle body of FIG. 12;

FIG. 14 is a plan view of a needle body according to a seventh modification;

FIG. 15A is a plan view of a needle body according to an eighth modification, and FIG. 15B is a cross-sectional view taken along line XVB-XVB of FIG. 15A;

FIG. 16A is a plan view of a needle body according to a ninth modification, and FIG. 16B is a cross-sectional view taken along line XVIB-XVIB in FIG. 16A;

FIG. 17A is a plan view of a needle body according to a tenth modification, FIG. 17B is a plan view of a needle body according to an eleventh modification, and FIG. 17C is a plan view of a needle body according to a twelfth modification;

FIG. 18 is an explanatory view illustrating an example of radiating light from a proximal end opening of a needle body; and

FIG. 19 is an explanatory view illustrating an example of radiating light from a distal end opening of a needle body.

### DETAILED DESCRIPTION

Hereinafter, preferred embodiments of a puncture needle, a catheter assembly, and a vascular puncture system according to the present invention will be described with reference to the accompanying drawings.

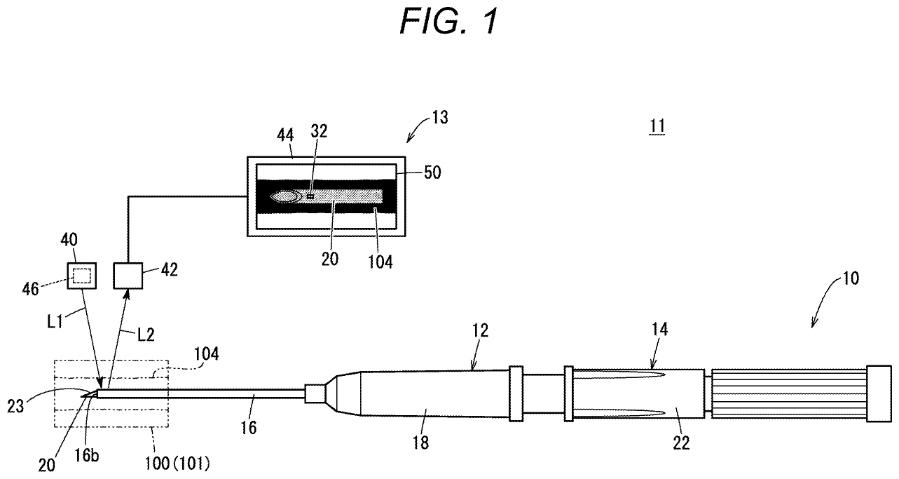

As illustrated in FIG. 1, a vascular puncture system 11 according to an embodiment of the present invention includes a catheter assembly 10 capable of puncturing a blood vessel 104 of a living body 100 (puncture target site 101), and a visualization device 13 for visualizing the blood vessel 104 and the catheter assembly 10 in the puncture target site 101.

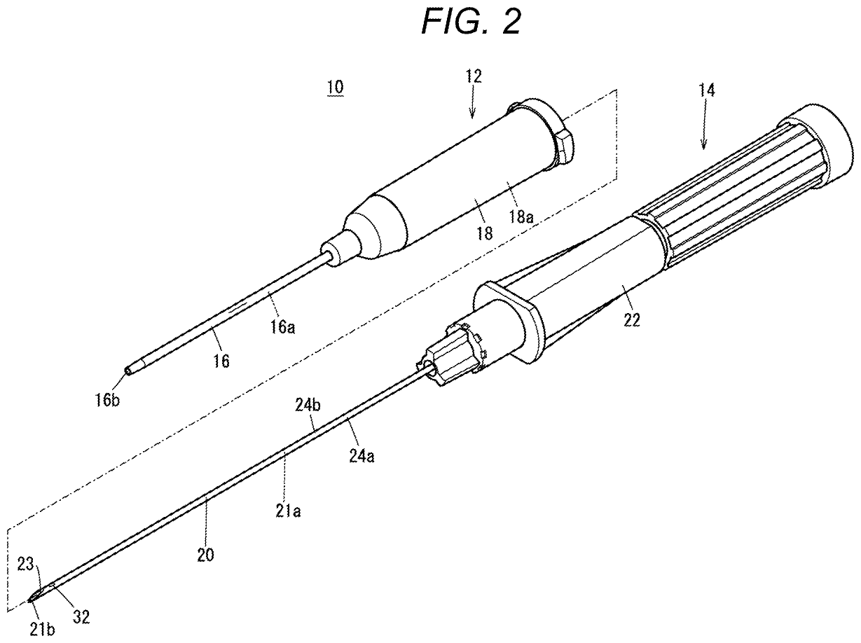

The catheter assembly 10 is configured as an indwelling needle for administering an infusion (drug) into a blood vessel 104 of a living body 100 (patient). However, the catheter assembly 10 is not limited to one that administers a drug. As illustrated in FIGS. 1 and 2, the catheter assembly 10 includes a catheter member 12 and a puncture needle 14. The catheter member 12 includes a catheter shaft 16 and a catheter hub 18 provided at a proximal end portion of the catheter shaft 16.

The catheter shaft 16 is a tubular member having flexibility and capable of being continuously inserted into the blood vessel 104 of the patient. The catheter shaft 16 has a lumen 16a extending along the axial direction over the entire length thereof. The catheter shaft 16 has at its distal end a distal end opening 16b communicating with the lumen 16a.

A constituent material of the catheter shaft 16 is not particularly limited, but a resin material having transparency, particularly a soft resin material is suitable, and examples thereof include a fluorine-based resin such as polytetrafluoroethylene (PTFE), ethylene-tetrafluoroethylene copolymer (ETFE), and perfluoroalkoxy fluorine resin (PFA), an olefin-based resin such as polyethylene and polypropylene or a mixture thereof, polyurethane, polyester, polyamide, polyether nylon resin, a mixture of an olefin-based resin and an ethylene-vinyl acetate copolymer, and the like. The catheter shaft 16 is configured to transmit the light L1 from the visualization device 13.

The catheter hub 18 is formed in a hollow shape (cylindrical shape). The catheter hub 18 is preferably made of a material harder than the catheter shaft 16. A constituent material of the catheter hub 18 is not particularly limited, but for example, thermoplastic resins such as polypropylene, polycarbonate, polyamide, polysulfone, polyarylate, a methacrylate-butylene-styrene copolymer, polyurethane, an acrylic resin, and an ABS resin can be suitably used.

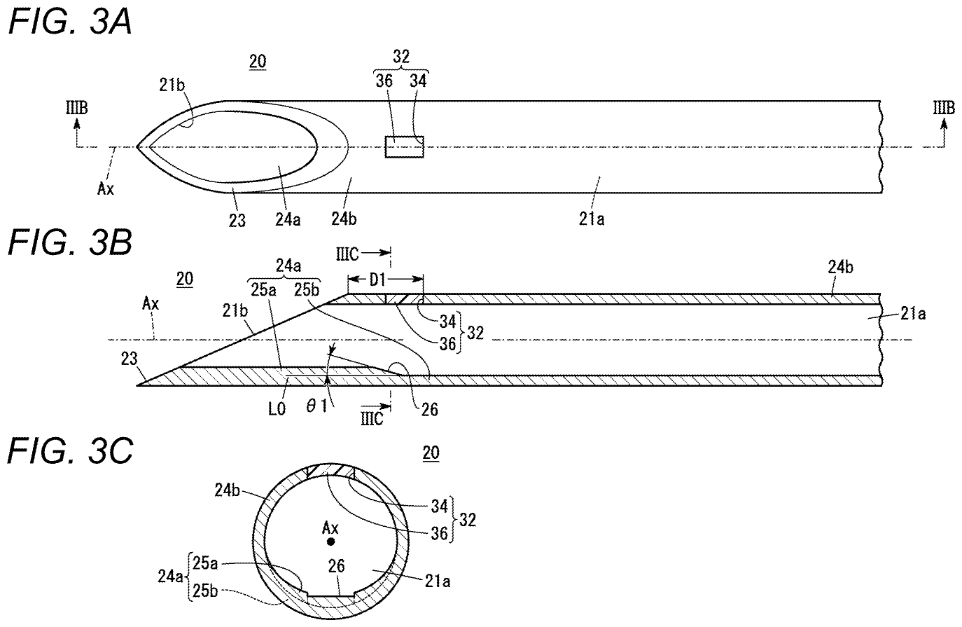

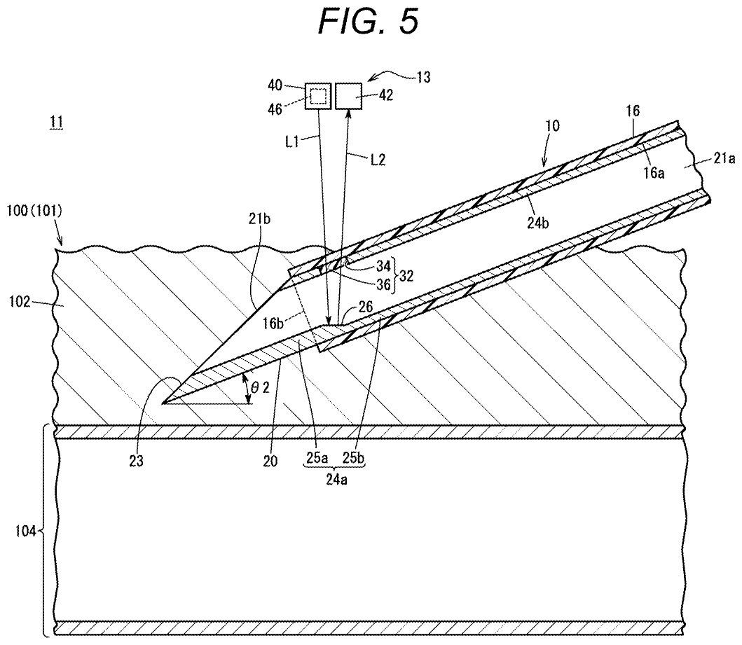

In FIG. 2, the puncture needle 14 includes a needle body 20 made of metal and a needle hub 22 provided at a proximal end portion of the needle body 20. As illustrated in FIGS. 2 to 3C, the needle body 20 is a tubular member having rigidity capable of puncturing a skin 102 (see FIG. 5) of the patient. Specifically, the needle body 20 is formed in a cylindrical shape. The needle body 20 has a lumen 21a extending along the axial direction. The needle body 20 is inserted into the lumen 16a of the catheter shaft 16 and a lumen 18a of the catheter hub 18 in the initial state (assembled state) of the catheter assembly 10 (see FIGS. 1 and 2).

Examples of the metal material constituting the needle body 20 include stainless steel, aluminum, an aluminum alloy, titanium, and a titanium alloy. The needle body 20 is formed sufficiently longer than the catheter shaft 16 and protrudes from the distal end opening 16b of the catheter shaft 16 in the initial state of the catheter assembly 10 (see FIG. 1).

In FIGS. 3A and 3B, the needle body 20 at its distal end portion a blade surface 23 inclined with respect to an axis Ax of the needle body 20. The blade surface 23 has a distal end opening 21b communicating with the lumen 21a of the needle body 20.

As illustrated in FIGS. 3B and 3C, the needle body 20 includes a first wall portion 24a located below the axis Ax of the needle body 20 in the horizontal state (state of FIG. 3B) of the needle body 20 in which the axis Ax of the needle body 20 is located in the horizontal direction such that the blade surface 23 faces upward, and a second wall portion 24b located above the axis Ax of the needle body 20 in the horizontal state of the needle body 20. In FIG. 3C, the first wall portion 24a forms the lower half of the needle body 20 in the horizontal state of the needle body 20. The first wall portion 24a extends 180° in the circumferential direction of the needle body 20. The second wall portion 24b forms the upper half of the needle body 20 in the horizontal state of the needle body 20. The second wall portion 24b extends 180° in the circumferential direction of the needle body 20. Both end portions of the first wall portion 24a are integrally connected to respective both end portions of the second wall portion 24b.

As illustrated in FIGS. 3B and 3C, the first wall portion 24a includes a thick portion 25a and a thin portion 25b. The thick portion 25a extends in the proximal direction from the distal end of the first wall portion 24a. The thin portion 25b extends from the proximal end of the thick portion 25a to the proximal end of the first wall portion 24a. One planar reflection portion 26 that reflects the light L1 from the visualization device 13 is formed on the inner face of the first wall portion 24a, (see FIG. 5). Specifically, the planar reflection portion 26 reflects at least one of near-infrared light and visible light. The planar reflection portion 26 is located at the distal end portion of the needle body 20.

The planar reflection portion 26 is provided at a position located at the lowermost portion of the thick portion 25a in the horizontal state of the needle body 20. The planar reflection portion 26 is a flat portion formed at the inner face of the needle body 20. In FIG. 3B, the planar reflection portion 26 is inclined radially outward of the needle body 20 from the distal end side toward the proximal end side of the needle body 20. The distal end of the planar reflection portion 26 is located on the proximal end side relative to the proximal end of the blade surface 23.

As illustrated in FIGS. 3A to 3C, one transmission window 32 is provided in the second wall portion 24b. In FIG. 3C, the transmission window 32 is provided at a position located at the uppermost portion of the second wall portion 24b in the horizontal state of the needle body 20. In the axial direction of the needle body 20, the distal end of the transmission window 32 is located between the distal end of the planar reflection portion 26 and the proximal end of the planar reflection portion 26. In the axial direction of the needle body 20, the proximal end of the transmission window 32 is located on a proximal end side relative to the proximal end of the planar reflection portion 26. That is, in top view of the needle body 20 in the horizontal state, part of the transmission window 32 overlaps part of the planar reflection portion 26. The distal end portion of the transmission window 32 faces the proximal end portion of the planar reflection portion 26. In other words, the distal end portion of the transmission window 32 is at a position shifted in phase by 180° in the circumferential direction of the needle body 20 with respect to the proximal end portion of the planar reflection portion 26.

In FIG. 3B, the transmission window 32 is located in the proximal direction relative to the proximal end of the blade surface 23. A distance D1 from the proximal end of the blade surface 23 to the proximal end of the transmission window 32 is within 2 mm. In other words, the transmission window 32 is located in a range within 2 mm in the proximal direction from the proximal end of the blade surface 23. The transmission window 32 is formed in a quadrangular shape. The shape of the transmission window 32 is not limited to the quadrangular shape.

As illustrated in FIGS. 3A to 3C, the transmission window 32 includes a through hole 34 penetrating the second wall portion 24b and a transmission member 36 disposed to close the through hole 34. In FIG. 5, the transmission member 36 is formed to be capable of transmitting the light L1 from the visualization device 13. In addition, the transmission window 32 is formed to be capable of transmitting the reflected light L2 of the light L1 guided from the visualization device 13 to the planar reflection portion 26. Specifically, the transmission member 36 is formed to be capable of transmitting at least one of near-infrared light and visible light. The transmission window 32 may not include the transmission member 36 and may be formed only of the through hole 34.

In FIG. 3B, an inclination angle θ1 of the planar reflection portion 26 with respect to a line segment L0 parallel to the axis Ax of the needle body 20 is 10° or more and 45° or less. In this case, as illustrated in FIG. 5, the inclination angle θ1 can be brought close to the puncture angle θ2 of the needle body 20 (the angle formed by the skin 102 and the needle body 20). That is, when the puncture target site 101 is punctured with the needle body 20, the planar reflection portion 26 can be brought into a state close to horizontal. Therefore, the light L1 guided from above the needle body 20 to the planar reflection portion 26 through the transmission window 32 can be efficiently reflected toward the transmission window 32. Note that the inclination angle θ1 can be appropriately changed according to the puncture angle θ2, and may be, for example, 15° or more and 30° or less.

In FIG. 3C, the outer peripheral surface of the needle body 20 is formed in an arc shape over the entire circumferential length in a cross section at a position of the planar reflection portion 26 in the needle body 20. That is, no step is formed on a portion of the outer peripheral surface of the needle body 20 on the back side of the planar reflection portion 26.

In FIGS. 1 and 2, the needle hub 22 is formed in a hollow shape (tubular shape). The constituent material of the needle hub 22 may be the same as the constituent material of the catheter hub 18 described above. A proximal end portion of the needle body 20 is fixed to a distal end portion of the needle hub 22. The needle hub 22 functions as an operation unit of the catheter assembly 10.

As illustrated in FIG. 1, the visualization device 13 includes an irradiation unit 40, a light receiving unit 42, and an image display unit 44. The irradiation unit 40 is disposed above the puncture target site 101 of the living body 100. The irradiation unit 40 irradiates the puncture target site 101 punctured with the needle body 20 with light L1 from above. The irradiation unit 40 includes a light source 46 that emits the light L1. The light L1 is near-infrared light. The near-infrared light includes, for example, wavelengths of 700 nm or more and 2500 nm or less, preferably 700 nm or more and 1400 nm or less, and more preferably 780 nm or more and 940 nm or less. Such light L1 is absorbed by blood and reflected by the metal needle body 20. The light source 46 may emit visible light (not including near-infrared light). Furthermore, the light source 46 may emit light including both near-infrared light and visible light.

The light receiving unit 42 is disposed above the puncture target site 101. That is, the light receiving unit 42 is located on the same side as the irradiation unit 40 with respect to the puncture target site 101. The light receiving unit 42 is a camera (imaging unit) that receives the reflected light L2 of the light L1 with which the irradiation unit 40 irradiates the puncture target site 101 and images the puncture target site 101 and the needle body 20. For example, the light receiving unit 42 includes a near-infrared CCD camera or the like. The image display unit 44 displays an image (reflected light image 50) created based on the reflected light L2 received by the light receiving unit 42.

Next, a method of forming the planar reflection portion 26 of the needle body 20 described above will be described.

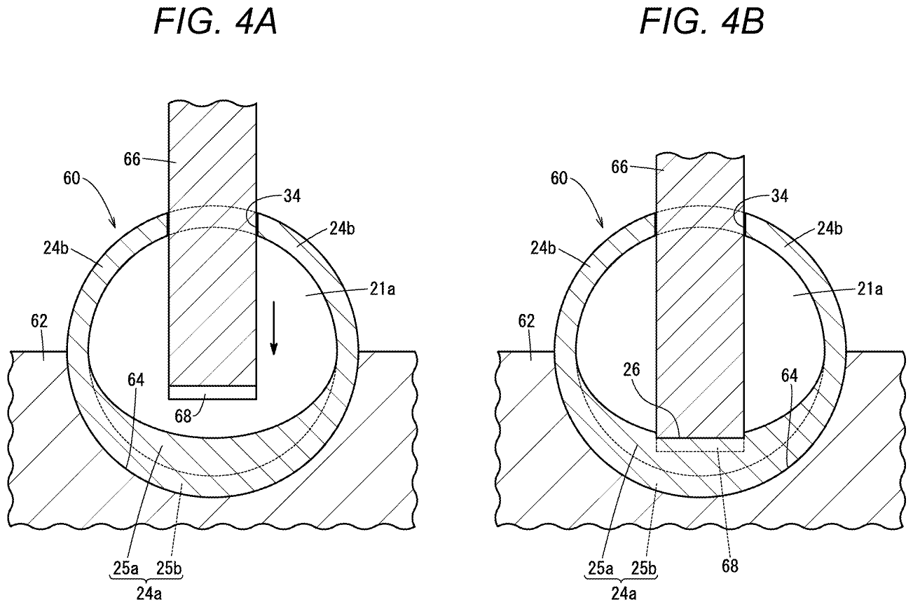

As illustrated in FIG. 4A, first, a metal cylindrical body 60 having a through hole 34 is prepared. The cylindrical body 60 includes a first wall portion 24a and a second wall portion 24b. The transmission member 36 is not yet provided in the through hole 34.

Then, the cylindrical body 60 is processed by a first member 62 and a second member 66. The first member 62 is formed in a block shape. The first member 62 has a first contact surface 64 that is in contact with the outer peripheral surface of the first wall portion 24a of the cylindrical body 60. The first contact surface 64 is a concave face having a shape (arc shape) corresponding to the outer peripheral surface of the first wall portion 24a. The second member 66 is, for example, a bar-shaped member having a quadrangular cross section (a shape corresponding to the through hole 34), and is passed through the through hole 34. However, the cross-sectional shape of the second member 66 is not limited to the quadrangular shape, and may be a circular shape or the like. The second member 66 has its distal end portion a second contact surface 68 having a planar shape and being in contact with the inner surface of the first wall portion 24a of the cylindrical body 60. The second contact face 68 is inclined with respect to the axis of the cylindrical body 60.

When the cylindrical body 60 is processed, first, the outer peripheral surface of the first wall portion 24a of the cylindrical body 60 is brought into contact with the first contact surface 64 of the first member 62. As a result, the cylindrical body 60 is supported by the first member 62. Then, as illustrated in FIG. 4B, the second member 66 is made to pass through the through hole 34 of the cylindrical body 60, and the second contact surface 68 is pressed against the inner surface of the first wall portion 24a of the cylindrical body 60. Then, the inner surface of the first wall portion 24a is plastically deformed into a shape (planar shape) corresponding to the second contact surface 68. That is, the planar reflection portion 26 is formed on the inner surface of the first wall portion 24a.

Next, a procedure of blood vessel puncture using the vascular puncture system 11 will be described.

As illustrated in FIG. 1, in the initial state of the catheter assembly 10, the blade surface 23 protrudes in the distal direction from the distal end opening 16b of the catheter shaft 16 in a state of facing upward.

First, the user sets the visualization device 13. Specifically, as illustrated in FIG. 5, the irradiation unit 40 and the light receiving unit 42 are disposed above the puncture target site 101 of the living body 100. Then, the irradiation unit 40 irradiates the puncture target site 101 with the light L1, and the needle body 20 (distal end portion of the catheter assembly 10) punctures the puncture target site 101.

Then, the light L1 emitted from the irradiation unit 40 is reflected by the puncture target site 101 and the needle body 20. At this time, the light L1 is absorbed by blood (hemoglobin) in the blood vessel 104 of the puncture target site 101. Then, the light receiving unit 42 receives the reflected light L2 reflected by the puncture target site 101 and the needle body 20 among the light L1.

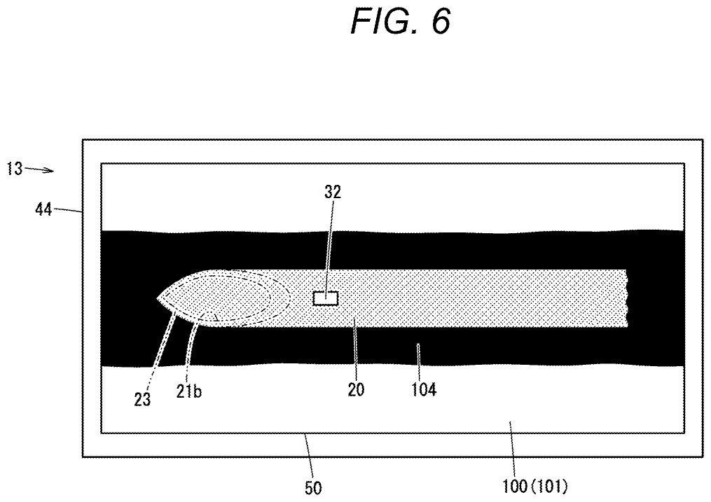

As a result, as illustrated in FIG. 6, a reflected light image 50 created based on the reflected light L2 received by the light receiving unit 42 is displayed on the image display unit 44. In the reflected light image 50, the blood vessel 104 and the needle body 20 in the puncture target site 101 are displayed. Specifically, for example, the blood vessel 104 and the needle body 20 are displayed in black in the reflected light image 50. In this case, since the color densities of the blood vessel 104 and the needle body 20 are different from each other, the user can distinguish the blood vessel 104 and the needle body 20 in the reflected light image 50.

In FIG. 5, the light L1 transmitted through the transmission window 32 of the needle body 20 is guided to the lumen 21a of the needle body 20. Of the light L1 guided to the lumen 21a of the needle body 20, the reflected light L2 reflected by the planar reflection portion 26 is transmitted through the transmission window 32, led above the puncture target site 101, and received by the light receiving unit 42. Therefore, as illustrated in FIG. 6, one transmission window 32 is displayed (for example, transmission window 32 is displayed in white) in the needle body 20 in the reflected light image 50.

At this time, as illustrated in FIG. 5, when the blade surface 23 is located above (directly above) the blood vessel 104, blood does not flow into the lumen 21a of the needle body 20. Therefore, in the reflected light image 50, the transmission window 32 does not change (for example, all transmission windows 32 remain white). As a result, the user can recognize that the needle body 20 is in the blood vessel unsecured state (state in which the distal end opening 21b of the needle body 20 is not located in the blood vessel 104) by visually recognizing the reflected light image 50 (state in which the transmission window 32 is not changed).

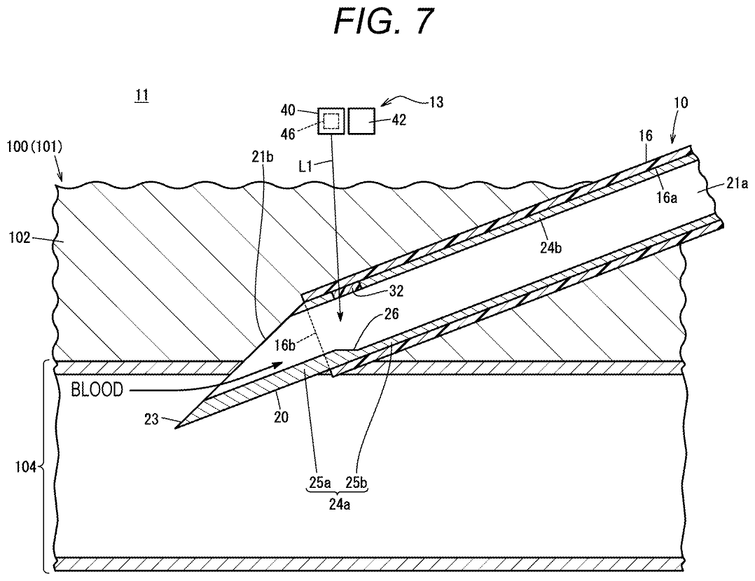

Subsequently, for example, the user operates the catheter assembly 10 to adjust the position of the blade surface 23. Then, as illustrated in FIG. 7, when the distal end opening 21b of the needle body 20 is inserted into the blood vessel 104, the blood in the blood vessel 104 flows from the distal end opening 21b of the needle body 20 into the lumen 21a of the needle body 20. When the transmission window 32 is covered with the blood in the lumen 21a of the needle body 20, the light L1 guided to the lumen 21a of the needle body 20 is absorbed by the blood before being guided to the planar reflection portion 26. Therefore, the light L1 of the lumen 21a of the needle body 20 is not led out from the transmission window 32.

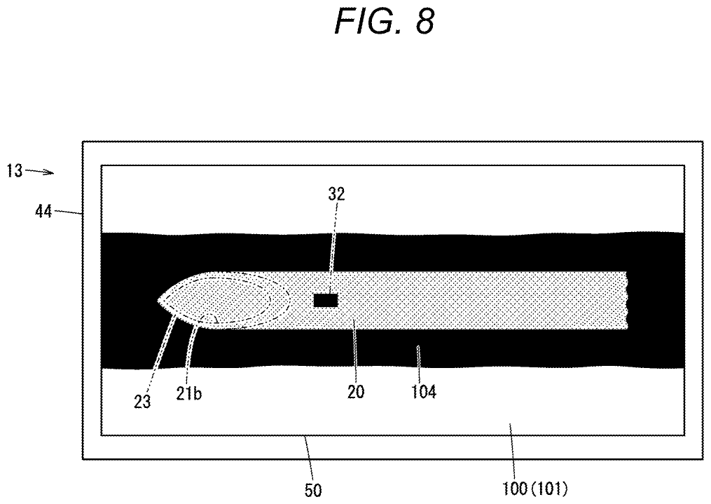

Therefore, as illustrated in FIG. 8, the appearance of the transmission window 32 of the needle body 20 changes in the reflected light image 50 (for example, the color changes from white to black). Therefore, the user can recognize that the needle body 20 is in the blood vessel secured state at an early stage before the blood is guided to the needle hub 22 by visually recognizing the reflected light image 50.

After securing the blood vessel of the needle body 20, the user removes the puncture needle 14 in a state where the distal end portion of the catheter shaft 16 is indwelled in the blood vessel 104, and administers a drug into the blood vessel 104 via the catheter shaft 16.

The puncture needle 14, the catheter assembly 10, and the vascular puncture system 11 according to the present embodiment have the following effects.

The needle body 20 includes a blade surface 23 formed at the distal end portion of the needle body 20, a planar reflection portion 26 provided on the inner surface of the needle body 20 to reflect the light L1, and a transmission window 32 capable of transmitting the reflected light L2 reflected by the planar reflection portion 26. The transmission window 32 is located on a proximal end side relative to the blade surface 23.

According to such a configuration, the light L1 with which the puncture target site 101 punctured by the needle body 20 is irradiated is transmitted through the transmission window 32 of the needle body 20 and is guided to the lumen 21a of the needle body 20. When the needle body 20 is in the blood vessel unsecured state and the blood does not flow into the lumen 21a of the needle body 20, the light L1 guided to the lumen 21a of the needle body 20 is reflected by the planar reflection portion 26. The reflected light L2 from the planar reflection portion 26 is transmitted through the transmission window 32 and is led to the outside of the needle body 20. Therefore, the user can visually recognize the transmission window 32 in the needle body 20 in the reflected light image 50.

On the other hand, when the needle body 20 is in the blood vessel secured state and the blood flowing into the lumen 21a of the needle body 20 covers the transmission window 32, the light L1 guided to the lumen 21a of the needle body 20 is absorbed by the blood and thus is not led from the transmission window 32 to the outside of the needle body 20. Therefore, the appearance of the transmission window 32 of the needle body 20 changes in the reflected light image 50. Therefore, the user can recognize the securing of the blood vessel of the needle body 20 based on the reflected light image 50.

The blade surface 23 is inclined with respect to the axis Ax of the needle body 20. The needle body 20 includes the first wall portion 24a located below the axis Ax of the needle body 20 in the horizontal state of the needle body 20 in which the axis Ax of the needle body 20 is located in the horizontal direction such that the blade surface 23 faces upward, and the second wall portion 24b located above the axis Ax of the needle body 20 in the horizontal state. The transmission window 32 is provided in the second wall portion 24b.

According to such a configuration, the reflected light L2 from the planar reflection portion 26 can be led above the needle body 20 via the transmission window 32.

The planar reflection portion 26 is provided on part of the inner surface of the first wall portion 24a in the longitudinal direction of the needle body 20.

According to such a configuration, the reflected light L2 from the planar reflection portion 26 can be efficiently guided to the transmission window 32.

The planar reflection portion 26 and the transmission window 32 are provided so as to face each other.

According to such a configuration, the reflected light L2 from the planar reflection portion 26 can be more efficiently guided to the transmission window 32.

The transmission window 32 is formed to be capable of transmitting the light L1 such that the light L1 is introduced into the lumen 21a of the needle body 20 from the outside of the needle body 20.

According to such a configuration, the light L1 can be introduced from the transmission window 32 into the lumen 21a of the needle body 20.

The planar reflection portion 26 is inclined radially outward of the needle body 20 toward the proximal direction of the needle body 20.

According to such a configuration, when the puncture is performed in a state where the needle body 20 is inclined by the predetermined puncture angle θ2 with respect to the puncture target site 101, the planar reflection portion 26 can be brought into a state close to horizontal. As a result, the reflected light L2 from the planar reflection portion 26 can be efficiently guided to the transmission window 32.

In the cross section at a position of the planar reflection portion 26 in the needle body 20, the outer peripheral surface of the needle body 20 is formed in an arc shape over the entire circumference.

According to such a configuration, since the step is not formed at the portion of the planar reflection portion 26 on the back side in the outer peripheral surface of the needle body 20, it is possible to suppress an increase in puncture resistance of the needle body 20 with respect to the puncture target site 101.

The transmission window 32 is provided in a range within 2 mm in the proximal direction from the proximal end of the blade surface 23 in the axial direction of the needle body 20.

According to such a configuration, it is possible to change the appearance of the transmission window 32 of the reflected light image 50 at a relatively early stage after the blood flows into the lumen 21a of the needle body 20 from the distal end opening 21b of the needle body 20.

The transmission window 32 can transmit near-infrared light.

According to such a configuration, the blood vessel 104 and the needle body 20 can be clearly displayed in the reflected light image 50.

The transmission window 32 includes the through hole 34 formed in the needle body 20 and the transmission member 36 disposed to close the through hole 34.

According to such a configuration, it is possible to suppress the blood in the lumen 21a of the needle body 20 flowing out of the transmission window 32 to the outside of the needle body 20.

The vascular puncture system 11 includes the puncture needle 14, the irradiation unit 40 for irradiating the puncture target site 101 punctured with the needle body 20 with light L1, and the light receiving unit 42 for receiving reflected light L2 reflected by the puncture target site 101.

According to such a configuration, the reflected light image 50 can be obtained by the irradiation unit 40 and the light receiving unit 42.

Each of the irradiation unit 40 and the light receiving unit 42 is disposed above the puncture target site 101 and the needle body 20.

According to such a configuration, it is possible to easily radiate and receive the light L1 to and from the puncture target site 101 and the needle body 20.

### First Modification

Next, a needle body 20a according to a first modification of the present invention will be described. In the needle body 20a according to the present modification, the same components as those of the needle body 20 described above are denoted by the same reference numerals, and a detailed description thereof will be omitted. In addition, in the needle body 20a according to the present modification, the same configuration as the above-described needle body 20 has the same effect. The same applies to needle bodies 20b to 201 according to second to twelfth modifications described later.

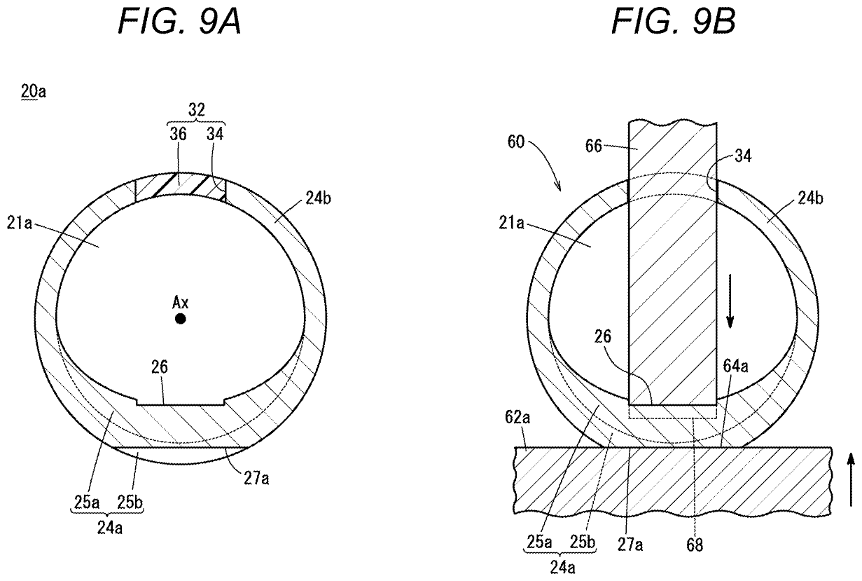

As shown in FIG. 9A, an outer flat portion 27a extending in parallel along the axial direction of the needle body 20a is formed at a portion, of the outer peripheral surface of the needle body 20a, on a back side of the planar reflection portion 26. In other words, the outer flat portion 27a is formed in the thick portion 25a of the first wall portion 24a. The outer flat portion 27a may extend parallel to the planar reflection portion 26.

As illustrated in FIG. 9B, the outer flat portion 27a of the needle body 20a is formed by processing the cylindrical body 60 with a first member 62a and the second member 66. The first contact surface 64a of the first member 62a is formed in a planar shape.

Specifically, a first contact surface 64a of the first member 62a is pressed against the outer peripheral surface of the first wall portion 24a of the cylindrical body 60, and the second contact surface 68 of the second member 66 is pressed against the inner surface of the first wall portion 24a of the cylindrical body 60. As a result, the outer peripheral surface of the first wall portion 24a is plastically deformed into a shape (planar shape) corresponding to the first contact surface 64a, and the inner surface of the first wall portion 24a is plastically deformed into a shape (planar shape) corresponding to the second contact surface 68. That is, the outer flat portion 27a is formed at the outer peripheral surface of the first wall portion 24a, and the planar reflection portion 26 is formed at the inner surface of the first wall portion 24a.

In the present modification, the outer flat portion 27a is formed at a portion, of the outer peripheral surface of the needle body 20a, on the back side of the planar reflection portion 26.

According to such a configuration, the planar reflection portion 26 can be easily formed at the inner surface of the needle body 20a using the first member 62a having the first contact surface 64a formed in a planar shape.

### Second Modification

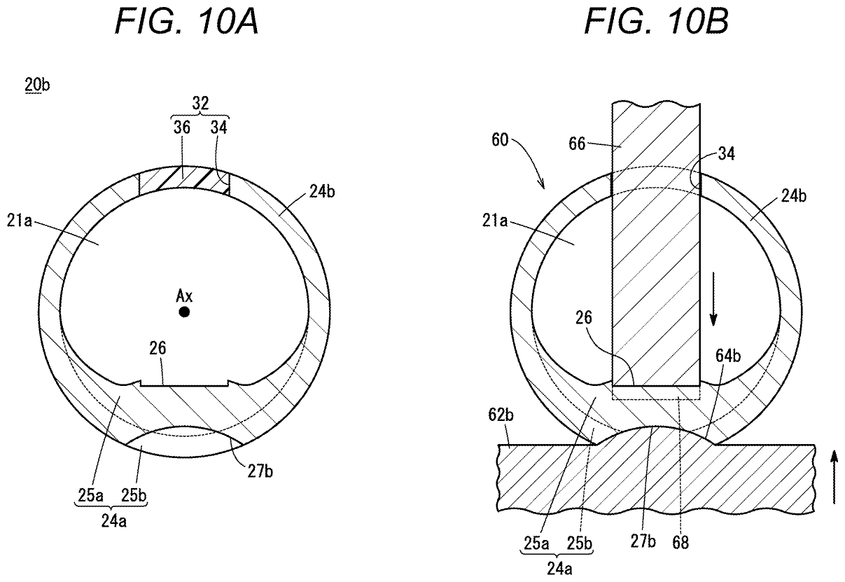

Next, a needle body 20b according to a second modification of the present invention will be described. As illustrated in FIG. 10A, a concave face portion 27b curved radially inward of the needle body 20b is formed at a portion, of the outer peripheral surface of the needle body 20b, on a back side of the planar reflection portion 26. The concave face portion 27b is formed in an arc shape.

As illustrated in FIG. 10B, such a needle body 20b is formed by processing the cylindrical body 60 with a first member 62b and the second member 66. The first contact surface 64b of the first member 62b is convexly curved.

Specifically, a first contact surface 64b of the first member 62b is pressed against the outer peripheral surface of the first wall portion 24a of the cylindrical body 60, and the second contact surface 68 of the second member 66 is pressed against the inner surface of the first wall portion 24a of the cylindrical body 60. Then, the outer peripheral surface of the first wall portion 24a is plastically deformed into a shape (concave curved surface) corresponding to the first contact surface 64b, and the inner surface of the first wall portion 24a is plastically deformed into a shape (planar shape) corresponding to the second contact surface 68. That is, the concave surface portion 27b is formed at the outer peripheral surface of the first wall portion 24a, and the planar reflection portion 26 is formed at the inner surface of the first wall portion 24a.

In the present modification, the concave surface portion 27b is formed at a portion, of the outer peripheral surface of the needle body 20b, on the back side of the planar reflection portion 26.

According to such a configuration, the planar reflection portion 26 can be easily formed at the inner surface of the needle body 20b using the first member 62b having the first contact surface 64b curved in a convex shape.

### Third Modification

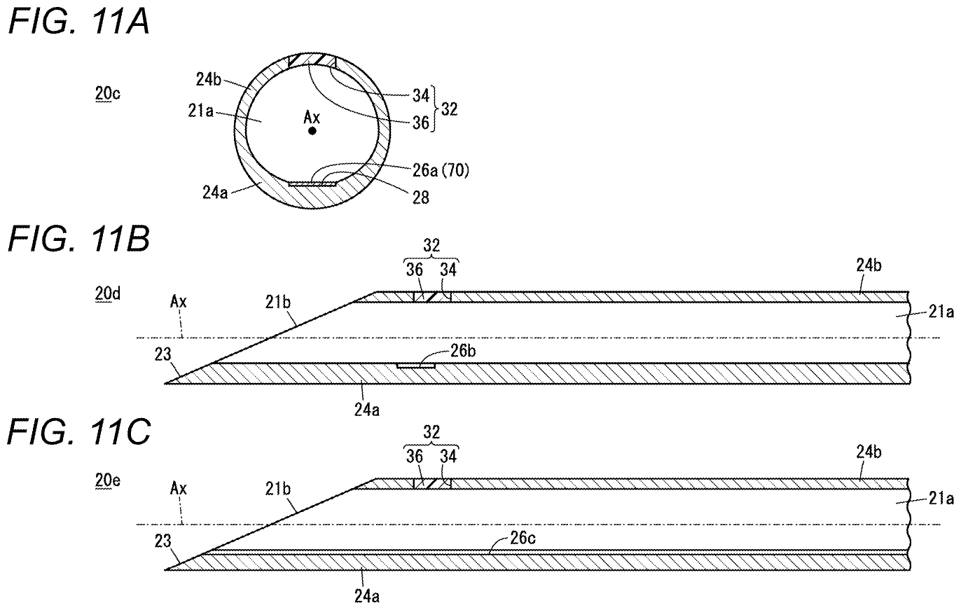

Next, a needle body 20c according to a third modification of the present invention will be described. As illustrated in FIG. 11A, in the needle body 20c, a planar reflection portion 26a is formed of a reflection member 70 provided on the inner surface of the needle body 20c. The reflection member 70 is provided on a flat portion 28 formed at the inner surface of the first wall portion 24a of the needle body 20c. The flat portion 28 is formed in the same manner as the above-described planar reflection portion 26. The reflectance of the reflection member 70 with respect to the light L1 from the visualization device 13 is higher than the reflectance of the needle body 20c with respect to the light L1 from the visualization device 13.

Specifically, the reflection member 70 is made of aluminum, silver, cyanine dye, or the like. The reflection member 70 may be a plate-shaped member. In this case, the reflection member 70 can be formed of, for example, a glass bead, a microprism, a corner cube mirror, or the like. The plate-shaped member is fixed to the flat portion 28 with an adhesive (not illustrated). The reflection member 70 may be formed by coating the flat portion 28 with a radiation scattering pigment, a radiation scattering dye, a fluororesin (FEP, PTFE), or the like.

In the present modification, the planar reflection portion 26a is formed of the reflection member 70 provided on the inner surface of the needle body 20c.

According to such a configuration, the light L1 guided to the lumen 21a of the needle body 20c can be effectively reflected by the planar reflection portion 26a.

### Fourth Modification

Next, a needle body 20d according to a fourth modification of the present invention will be described. As illustrated in FIG. 11B, a planar reflection portion 26b extending in parallel to the axis Ax of the needle body 20d is formed at the inner surface of the first wall portion 24a of the needle body 20d. The first wall portion 24a has a substantially constant thickness over the entire length. The planar reflection portion 26b is formed only at part of the needle body 20d in the axial direction. The planar reflection portion 26b faces the transmission window 32.

In the present modification, planar reflection portion 26b extends parallel to the axis Ax of the needle body 20d.

According to such a configuration, the planar reflection portion 26b can be easily formed at the inner surface of the needle body 20d.

### Fifth Modification

Next, a needle body 20e according to a fifth modification of the present invention will be described. As illustrated in FIG. 11C, a planar reflection portion 26c extending in parallel to the axis Ax of the needle body 20e is formed at the inner surface of the first wall portion 24a of the needle body 20e. The first wall portion 24a has a substantially constant thickness over the entire length. The planar reflection portion 26c is provided over the entire length of the needle body 20e in the axial direction. The planar reflection portion 26c faces the transmission window 32. The needle body 20e according to the present modification has the same effect as the needle body 20d according to the fourth modification described above.

### Sixth Modification

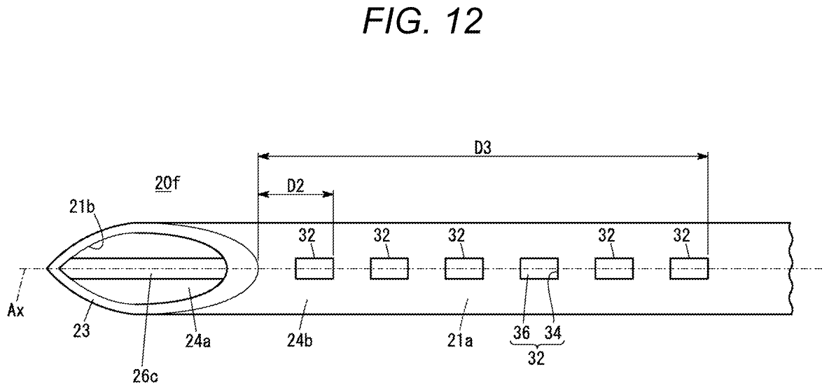

Next, a needle body 20f according to a sixth modification of the present invention will be described. As illustrated in FIG. 12, in the needle body 20f, a plurality of (6 in the example of FIG. 12) transmission windows 32 is provided. The plurality of transmission windows 32 is provided at equal intervals along the axial direction of the needle body 20f. In top view of the needle body 20f in the horizontal state, the axis Ax of the needle body 20f passes through the center of each transmission window 32 in the circumferential direction of the needle body 20f. In other words, each transmission window 32 is provided on the axis Ax of the needle body 20f in top view of the needle body 20f in the horizontal state. The number of transmission windows 32 is not limited to 6. The plurality of transmission windows 32 each is provided at a position located at the uppermost portion of the second wall portion 24b in the horizontal state of the needle body 20f.

The transmission window 32 located on the most distal end side of the needle body 20f is located in the proximal direction relative to the proximal end of the blade surface 23. A distance D2 from the proximal end of the blade surface 23 to the proximal end of the transmission window 32 located on the most distal end side of the needle body 20f is within 2 mm. In other words, the transmission window 32 located on the most distal end side of the needle body 20f is located in a range within 2 mm in the proximal direction from the proximal end of the blade surface 23. Note that two or more transmission windows 32 may be disposed in a range within 2 mm in the proximal direction from the proximal end of the blade surface 23 in the needle body 20f.

A distance D3 from the proximal end of the blade surface 23 to the proximal end of the transmission window 32 located on the most proximal end side of the needle body 20f is within 30 mm. In other words, all of the plurality of transmission windows 32 are located in a range within 30 mm in the proximal direction from the proximal end of the blade surface 23. However, the transmission window 32 may be provided in a range exceeding 30 mm in the proximal direction from the proximal end of the blade surface 23, or may be provided at up to the proximal end portion of the needle body 20f.

The plurality of transmission windows 32 is formed to have the same size and the same shape. However, the plurality of transmission windows 32 may be formed in different sizes or may be formed in different shapes.

However, the needle body 20f includes the above-described planar reflection portion 26c. However, the needle body 20f may include a plurality of planar reflection portions 26, 26a, 26b that faces the plurality of transmission windows 32.

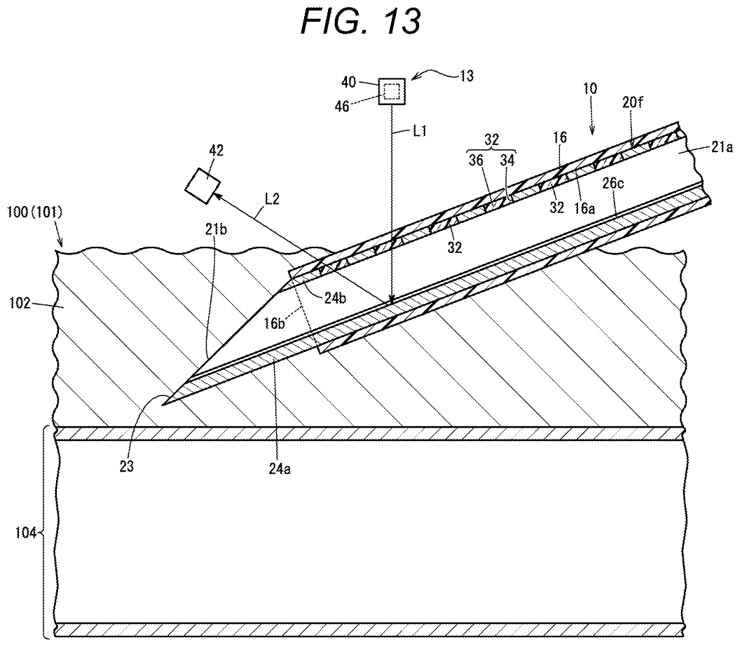

In the present modification, as illustrated in FIG. 13, the transmission window 32 through which the light L1 incident on the lumen 21a of the needle body 20f is transmitted and the transmission window 32 through which the reflected light L2 reflected by the planar reflection portion 26c is transmitted may be different from each other.

In the present modification, a plurality of transmission windows 32 is provided along the axis Ax of the needle body 20f.

According to such a configuration, a change in the transmission window 32 can be easily seen in the reflected light image 50.

### Seventh Modification

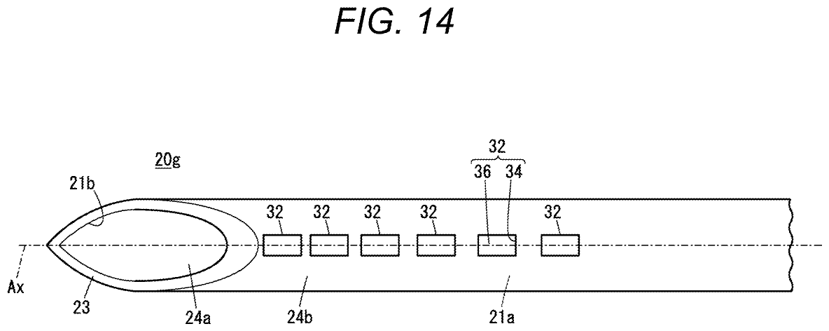

Next, a needle body 20g according to a seventh modification of the present invention will be described. As illustrated in FIG. 14, a plurality of (6 in the example of FIG. 14) transmission windows 32 is provided in the needle body 20g. In the needle body 20g, the distance between the transmission windows 32 adjacent to each other on the distal end side of the needle body 20g is narrower than the distance between the transmission windows 32 adjacent to each other on the proximal end side of the needle body 20g. In other words, the distance between the transmission windows 32 adjacent to each other gradually increases from the distal end side toward the proximal end side.

In top view of the needle body 20g in the horizontal state, the axis Ax of the needle body 20g passes through the center of each transmission window 32 in the circumferential direction of the needle body 20g. In other words, each transmission window 32 is provided on the axis Ax of the needle body 20g in top view of the needle body 20g in the horizontal state. The number of transmission windows 32 is not limited to 6. The plurality of transmission windows 32 each is provided at a position located at the uppermost portion of the second wall portion 24b in the horizontal state of the needle body 20g.

The needle body 20g includes at its inner surface of the plurality of planar reflection portions 26 that faces the plurality of transmission windows 32. However, the needle body 20g may be provided with the above-described planar reflection portions 26a to 26c.

According to such a configuration, it is possible to easily visually recognize the change in the transmission window 32 in the reflected light image 50 at the initial stage when the blood flows into the lumen 21a of the needle body 20g. As a result, the blood vessel securing of the needle body 20g can be quickly and stepwise recognized.

### Eighth Modification

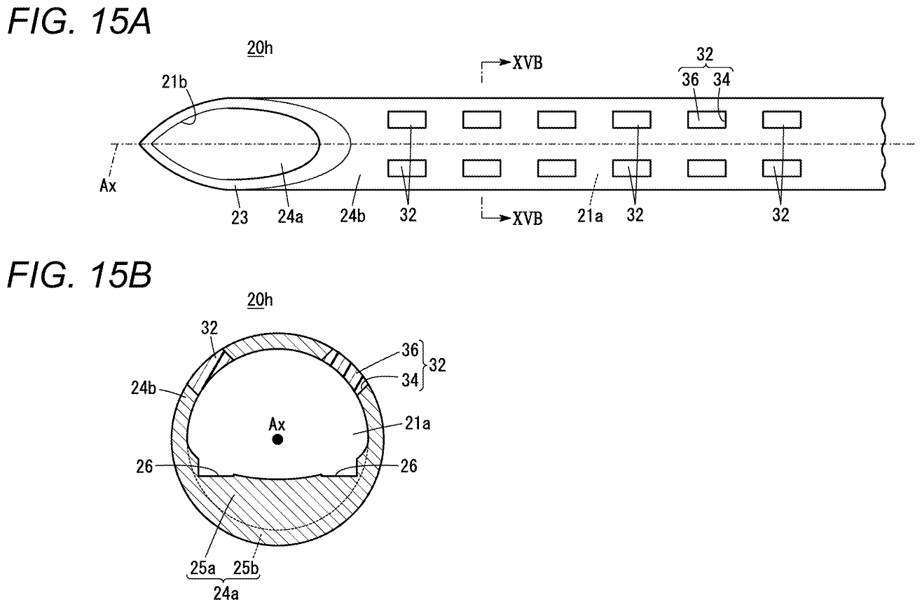

Next, a needle body 20h according to an eighth modification of the present invention will be described. As illustrated in FIGS. 15A and 15B, in the needle body 20h, the plurality of transmission windows 32 is disposed at equal intervals in the axial direction of the needle body 20h in a state of being disposed in two rows. Note that the plurality of transmission windows 32 is not limited to be disposed in two rows, and may be disposed at equal intervals in the axial direction of the needle body 20h in a state of being disposed in three or more rows.

The two transmission windows 32 adjacent to each other in the circumferential direction of the needle body 20h are located so as to sandwich the uppermost portion of the second wall portion 24b. Each transmission window 32 faces upward in the horizontal state of the needle body 20h. The distance between the two transmission windows 32 adjacent to each other in the circumferential direction of the needle body 20h can be appropriately set.

Note that the needle body 20h includes at its inner surface of the plurality of planar reflection portions 26 that faces the plurality of transmission windows 32. That is, the plurality of planar reflection portions 26 is disposed at equal intervals in the axial direction of the needle body 20h in a state of being disposed in two rows. However, the needle body 20h may be provided with the above-described planar reflection portions 26a to 26c.

According to such a configuration, it is possible to more easily visually recognize the change in the appearance of the transmission window 32 in the reflected light image 50.

### Ninth Modification

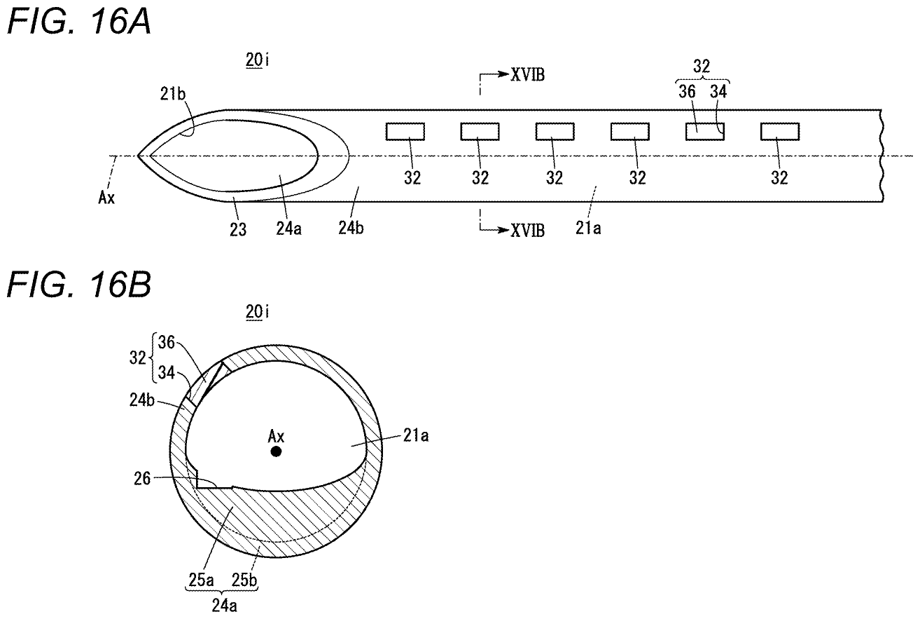

Next, a needle body 20i according to a ninth modification of the present invention will be described. As illustrated in FIGS. 16A and 16B, in the needle body 20i, the plurality of transmission windows 32 is provided at equal intervals along the axial direction of the needle body 20i. In top view of the needle body 20i in the horizontal state, each transmission window 32 is provided at a position shifted in the circumferential direction of the needle body 20i with respect to the axis Ax of the needle body 20i. That is, in top view of the needle body 20i in the horizontal state, each transmission window 32 does not overlap the axis Ax of the needle body 20i.

Note that the needle body 20i includes at its inner surface of the plurality of planar reflection portions 26 that faces the plurality of transmission windows 32. However, the needle body 20i may be provided with the above-described planar reflection portions 26a to 26c.

According to such a configuration, it is possible to easily visually recognize the change in the appearance of the transmission window 32 in the reflected light image 50.

### Tenth Modification

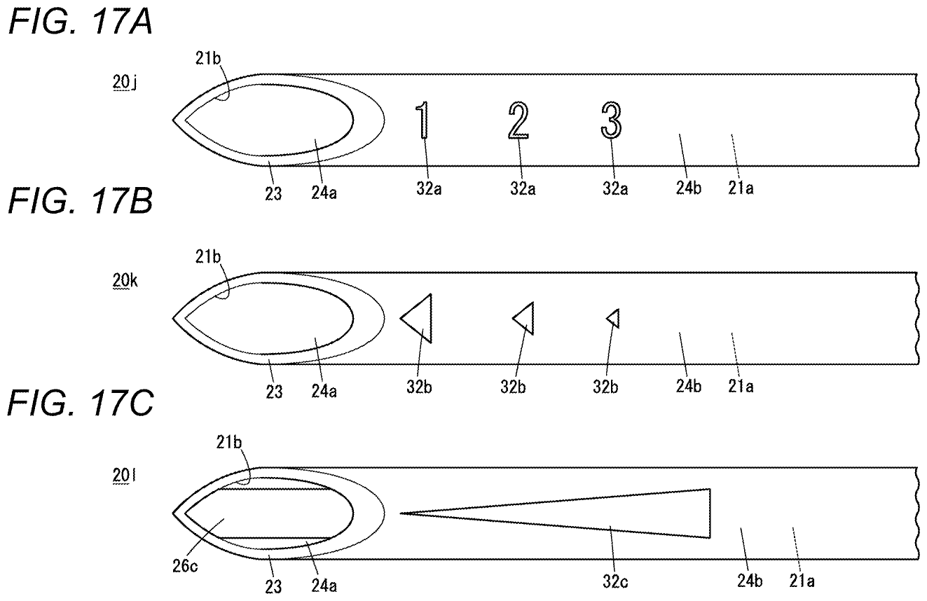

Next, a needle body 20j according to a tenth modification of the present invention will be described. As illustrated in FIG. 17A, in the needle body 20j, a plurality of (three in FIG. 17A) transmission windows 32a is formed in an Arabic figure shape. The transmission window 32a is formed so as to be disposed in the order of 1, 2, and 3 from the distal end side toward the proximal end side of the needle body 20j. However, the number of transmission windows 32a may be two or four or more. In addition, the arrangement of the numbers can be changed as appropriate. Furthermore, the transmission window 32a may be displayed with Roman numerals, Greek numerals, or the like.

Note that the needle body 20j includes at its inner surface of the plurality of planar reflection portions 26 that faces the plurality of transmission windows 32a. However, the needle body 20j may be provided with the above-described planar reflection portions 26a to 26c.

The plurality of transmission windows 32a is different from each other in shape.

According to such a configuration, a change in the transmission window 32a can be easily seen in the reflected light image 50.

### Eleventh Modification

Next, a needle body 20k according to an eleventh modification of the present invention will be described. As illustrated in FIG. 17B, in the needle body 20k, each transmission window 32b is formed in a triangular shape (regular triangular shape). One side of the triangle of the transmission window 32b extends along the circumferential direction of the needle body 20k. The size of the transmission window 32b gradually decreases from the distal end side toward the proximal end side of the needle body 20k. However, the sizes of the plurality of transmission windows 32b may be the same as each other. The shape of the transmission window 32b is not limited to the triangular shape, and may be a circular shape, an arrow shape, or the like.

Note that the needle body 20k includes at its inner surface of the plurality of planar reflection portions 26 that faces the plurality of transmission windows 32b. However, the needle body 20k may be provided with the above-described planar reflection portions 26a to 26c.

The plurality of transmission windows 32b is different from each other in size.

According to such a configuration, a change in the transmission window 32b can be easily seen in the reflected light image 50.

### Twelfth Modification

Next, a needle body 201 according to a twelfth modification of the present invention will be described. As illustrated in FIG. 17C, in the needle body 201, only one transmission window 32c is provided. The transmission window 32c is formed in a triangular shape (isosceles triangular shape). One side of the triangle of the transmission window 32c extends along the circumferential direction of the needle body 201. The width of the transmission window 32c in the circumferential direction of the needle body 201 gradually increases from the distal end side toward the proximal end side of the needle body 201. The shape of the transmission window 32c is not limited to the triangular shape, and may be a quadrangular shape, an elliptical shape, or the like.

Note that the needle body 201 includes at its inner surface of the planar reflection portion 26c that faces the transmission window 32c. However, the needle body 201 may be provided with the above-described planar reflection portions 26, 26a, and 26b.

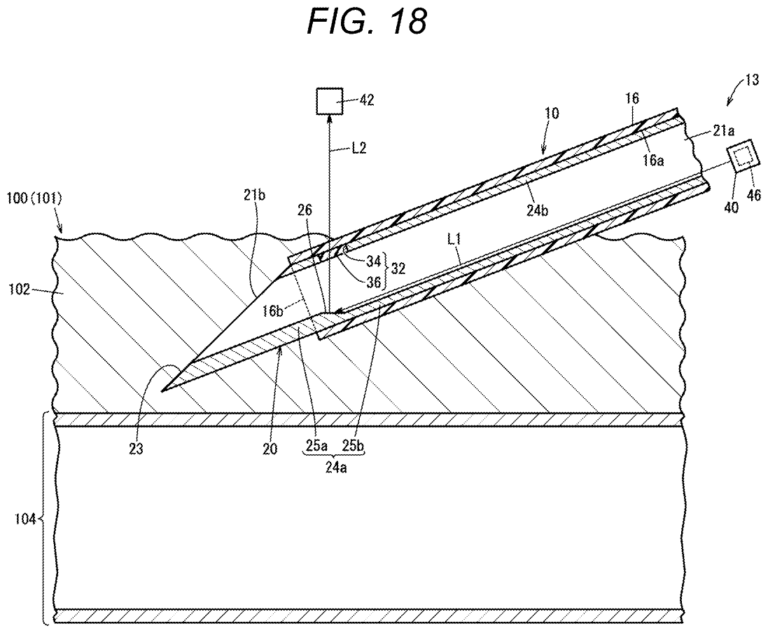

In the visualization device 13 of the vascular puncture system 11 described above, as illustrated in FIG. 18, the irradiation unit 40 may be disposed so as to capable of radiating the light L1 from the proximal end opening of the needle body 20 toward the planar reflection portion 26, and the light receiving unit 42 may be disposed above the transmission window 32. In this case, for example, it is preferable to use the needle body 20 provided with the planar reflection portion 26 inclined radially outward of the needle body 20 toward the proximal direction of the needle body 20. According to such a needle body 20, the light L1 guided from the proximal end side of the needle body 20 can be efficiently reflected to the transmission window 32 (upper side). However, the needle bodies 20a to 201 described above may be used in such an arrangement of the irradiation unit 40 and the light receiving unit 42.

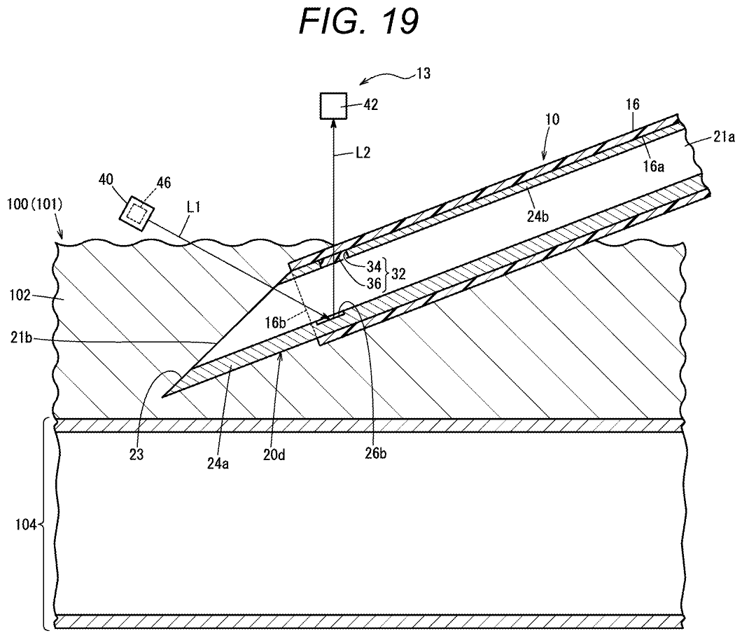

In addition, in the visualization device 13 of the vascular puncture system 11 described above, as illustrated in FIG. 19, the irradiation unit 40 may be disposed so as to be capable of radiating the light L1 from the distal end opening 21b of the needle body 20d toward the planar reflection portion 26b, and the light receiving unit 42 may be disposed above the transmission window 32. In this case, for example, it is preferable to use the needle body 20d provided with the planar reflection portion 26b extending parallel to the axial direction of the needle body 20d. According to such a needle body 20d, the light L1 guided from the distal end side of the needle body 20d can be efficiently reflected to the transmission window 32 (upper side). However, in such an arrangement of the irradiation unit 40 and the light receiving unit 42, the needle bodies 20, 20a to 20c, and 20e to 201 described above may be used.

The present invention is not limited to the above-described embodiments, and various modifications can be made without departing from the gist of the present invention.

The above embodiments are summarized as follows.

The above embodiment discloses a medical puncture needle (14) including a metal needle body (20, 20a to 20l) formed in a tubular shape, in which the needle body includes a blade surface (23) formed at a distal end portion of the needle body, a planar reflection portion (26, 26a to 26c) provided at an inner surface of the needle body to reflect light (L1), and a transmission window (32, 32a to 32c) capable of transmitting reflected light (L2) reflected by the planar reflection portion, and the transmission window is located on a proximal end side relative to the blade surface.

In the above-described puncture needle, the blade surface may be inclined with respect to an axis (Ax) of the needle body, the needle body may include a first wall portion (24a) positioned below the axis of the needle body in a horizontal state of the needle body in which the axis of the needle body is positioned in a horizontal direction such that the blade surface faces upward, and a second wall portion (24b) positioned above the axis of the needle body in the horizontal state, and the transmission window may be provided in the second wall portion.

In the above-described puncture needle, the planar reflection portion may be provided at the inner surface of the first wall portion over the entire length or part of the needle body in the longitudinal direction.

In the puncture needle, the planar reflection portion and the transmission window may be provided to face each other.

In the above-described puncture needle, the transmission window may be formed so as to be capable of transmitting the light such that the light is introduced into a lumen (21a) of the needle body from the outside of the needle body.

In the above-described puncture needle, the planar reflection portion may extend in parallel to the axis of the needle body.

In the above-described puncture needle, the planar reflection portion may be inclined radially outward of the needle body toward the proximal direction of the needle body.

In the above-described puncture needle, the outer peripheral surface of the needle body may be formed in an arc shape over the entire length in the circumferential direction in a cross section at a position of the planar reflection portion in the needle body.

In the above-described puncture needle, an outer flat portion (27a) or a concave face portion (27b) may be formed at a portion, of an outer peripheral face of the needle body, on a back side of the planar reflection portion.

In the above-described puncture needle, the planar reflection portion may be formed of a reflection member (70) provided at an inner surface of the needle body.

In the above-described puncture needle, a plurality of the transmission windows may be provided along the axis of the needle body.

In the above-described puncture needle, the plurality of transmission windows may be different from each other in at least one of a shape and a size.

In the above-described puncture needle, at least one of the transmission windows may be provided in a range within 2 mm in a proximal direction from a proximal end of the blade surface in the axial direction of the needle body.

In the puncture needle, the transmission window may transmit near-infrared light.

In the above-described puncture needle, the transmission window may include a through hole (34) formed in the needle body, and a transmission member (36) disposed to close the through hole.

The above embodiment discloses a catheter assembly (10) including the above-described puncture needle and a catheter shaft (16) having a lumen (16a) through which the needle body is inserted.

The above embodiment discloses a vascular puncture system (11) including the above-described puncture needle, an irradiation unit (40) for irradiating a puncture target site (101) punctured with the needle body with the light, and a light receiving unit (42) for receiving reflected light reflected by the puncture target site and the needle body.

In the above-described vascular puncture system, each of the irradiation unit and the light receiving unit may be disposed above the puncture target site and the needle body.

In the above-described vascular puncture system, the irradiation unit may be disposed to be capable of radiating the light from the distal end opening (21b) or the proximal end opening of the needle body toward the planar reflection portion, and the light receiving unit may be disposed above the transmission window.

What is claimed is:

1. A medical puncture needle comprising:
a metal needle body formed in a tubular shape, the needle body comprising:
a blade surface located at a distal end portion of the needle body,
a planar reflection portion located at an inner surface of the needle body and configured to reflect light, and
a transmission window located proximal of the blade surface and configured to transmit reflected light reflected by the planar reflection portion.

2. The puncture needle according to claim 1, wherein:
the blade surface is inclined with respect to an axis of the needle body;
the needle body comprises:
a first wall portion located below the axis of the needle body in a horizontal state of the needle body in which the axis of the needle body is located in a horizontal direction such that the blade surface faces upward, and
a second wall portion located above the axis of the needle body in the horizontal state; and
the transmission window is located in the second wall portion.

3. The puncture needle according to claim 2, wherein:
the planar reflection portion is located at an inner surface of the first wall portion over an entire length of the needle body in a longitudinal direction.

4. The puncture needle according to claim 2, wherein:
the planar reflection portion is located at an inner surface of the first wall portion over part of a length of the needle body in a longitudinal direction.

5. The puncture needle according to claim 2, wherein:
the planar reflection portion is located at an inner surface of the first wall portion; and
the planar reflection portion and the transmission window face each other.

6. The puncture needle according to claim 1, wherein:
the transmission window is configured to transmit the light so that the light is introduced into a lumen of the needle body from an outside of the needle body.

7. The puncture needle according to claim 1, wherein:
the planar reflection portion extends parallel to an axis of the needle body.

8. The puncture needle according to claim 1, wherein:
the planar reflection portion is inclined radially outward in a a proximal end direction of the needle body.

9. The puncture needle according to claim 1, wherein:
in a cross section at a position of the planar reflection portion in the needle body, an outer peripheral surface of the needle body is formed in an arc shape over an entire circumference.

10. The puncture needle according to claim 1, wherein:
an outer flat portion or a concave surface portion is formed at a portion of an outer peripheral face of the needle body on a back side of the planar reflection portion.

11. The puncture needle according to claim 1, wherein:
the planar reflection portion comprises a reflection member located at an inner surface of the needle body.

12. The puncture needle according to claim 1, wherein:
the transmission window comprises a plurality of the transmission windows arrayed along an axis of the needle body.

13. The puncture needle according to claim 12, wherein:
the plurality of transmission windows includes transmission windows that are different from each other in shape and/or size.

14. The puncture needle according to claim 1, wherein:
in the axial direction of the needle body, the transmission window is located at a distance in a range within 2 mm in a proximal end direction from a proximal end of the blade surface.

15. The puncture needle according to claim 1, wherein:
the transmission window is configured to transmit near-infrared light.

16. The puncture needle according to claim 1, wherein:
the transmission window comprises:
a through hole formed in the needle body, and
a transmission member disposed to close the through hole.

17. A catheter assembly comprising:
the puncture needle according to claim 1; and
a catheter shaft having a lumen through which the needle body is inserted.

18. A vascular puncture system comprising:
a medical puncture needle comprising:
a metal needle body formed in a tubular shape, the needle body comprising:
a blade surface located at a distal end portion of the needle body,
a planar reflection portion located at an inner surface of the needle body and configured to reflect light, and
a transmission window located proximal of the blade surface and configured to transmit reflected light reflected by the planar reflection portion;
an irradiation unit configured to irradiate a puncture target site punctured with the needle body with the light; and
a light receiving unit configured to receive reflected light reflected by the puncture target site and the needle body.

19. The vascular puncture system according to claim 18, wherein
each of the irradiation unit and the light receiving unit is configured to be disposed above the puncture target site and the needle body.

20. The vascular puncture system according to claim 18, wherein:
the irradiation unit is configured to radiate the light from a distal end opening or a proximal end opening of the needle body toward the planar reflection portion; and
the light receiving unit is configured to be disposed above the transmission window.

21. A method of determining whether a distal end opening of a needle body is located in a blood vessel, the method comprising:
providing a medical puncture needle comprising:
a metal needle body formed in a tubular shape, the needle body comprising:
a blade surface located at a distal end portion of the needle body and comprising a distal end opening,
a planar reflection portion located at an inner surface of the needle body and configured to reflect light, and
a transmission window located proximal of the blade surface and configured to transmit reflected light reflected by the planar reflection portion;
providing an irradiation unit at a position above a puncture target site of a living body;
providing a light receiving unit at a position above a puncture target site of a living body;
irradiating the puncture target site with light from the irradiation unit;
puncturing the puncture target site with the needle body;
receiving the reflected light reflected by the planar reflection portion at the light receiving unit;
adjusting a position of the blade surface; and
determining whether the distal end opening of the needle body is located in the blood vessel by viewing an image of the reflected light.
